(19) United States
(12) Patent Application Publication
(10) Pub. No.: US 2003/0208280 A1
(43) Pub. Date: Nov. 6, 2003

(54) Wear resistant artificial joint

(76) Inventor: Behrooz Tohidi, Rancho Santa Fe, CA (US)

Correspondence Address: Peter K Hahn, Luce Forward Hamilton & Scripps, Suite 2600, 600 West Broadway, San Diego, CA, 92101, US

(21) Appl. No.: 10/296,568

(22) PCT Filed: Apr. 20, 2001
(86) PCT No.: PCT/US01/12870

(51) Int. Cl. A61F 2/30

(52) U.S. Cl. 623/23.39; 623/23.4; 623/23.58

(57) ABSTRACT

Artificial joints wherein the mating surfaces are both non-metallic, preferably plastic, even more preferably polyethylene and even more preferably ultra-high molecular weight polyethylene. Because neither of the bearing surfaces of the artificial joint are made of metal, there is a potential for greatly reduced wear, and consequently, a potential for reducing the number of joint replacement surgeries.

The present invention relates to orthopedic prostheses utilized in joint replacements, and more particularly to prosthetic joint replacements wherein there is moving contact between mating (or bearing) prosthetic surfaces.

### BACKGROUND OF THE INVENTION

The bones of a human (or animal) skeleton provide rigid, structural reinforcement for the body. Despite this rigidity, movement of the body (walking, chewing, etc.) is still permitted by the fact that bones are attached to each other at movable joints, where two or more separate bones move relative to each other.

One common joint geometry is the hinge joint, such as the hinge joint of the human elbow or knee. A pure hinge joint allows relative rotation, such that both bones maintain a constant, mutual planar orientation. Another common joint geometry is the ball-and-socket joint, such as the human shoulder joint. A ball-and-socket joint allows rotation in more than one plane. Then there are more complex joint geometries, which can facilitate complex combinations of relative translational and rotational movement between adjacent bones. Examples of these more complex geometries can be found in the human jaw and the human hip.

In all of these types of joints, bones come into direct, physical contact with each other. At the joint, the bones move relative to each other, exhibiting rotational contact, rolling contact, translational contact, or some combination of these. Of course, this moving contact causes wear on the joints themselves.

However, the portions of bones that come into mutual contact at joints are generally coated with cartilaginous material (or cartilage). The natural cartilage has a combination of material properties, such as hardness, coefficients of friction, elasticity and compressive strength, so that the cartilage wear due to moving contact at the joint is relatively slow, and the cartilage at the joint typically outlasts the life of the human or animal to whom the joint belongs. In this case, the wear at the joint is not considered very problematic.

Nevertheless, some people live long enough that their joint cartilage deteriorates and/or wears out. More particularly, the cartilage can wear or deteriorate down to the bone material portion of the joint. When this happens, catastrophic or semi-catastrophic failure of the bone can occur due to the lack of capacity that bone matter has with respect to moving contact and wear. This wear problem is especially likely to occur if the joint: (1) had less cartilage than normal to begin with; (2) is subject to a disease that affects cartilage; (3) is subject to an accident or other trauma affecting the cartilage; (4) is exercised more often than average; and/or (5) is routinely placed under greater-than-average stress. The problem of cartilage wear is especially prevalent at the knee joint and hip joint, in part because the weight of the human body bears on this joint as it moves during walking, running and even standing, thereby exacerbating wear.

In an effort to restore functions of a deteriorated or damaged joint, surgeons have devised numerous methods of partial and total joint replacements. In these joint replacements, portions of the bone at the existing joint are either removed or supplemented with prosthetic components designed to restore some or all of the original functionality of the joint. Today, hip and knee joint replacements are common. Increasingly, shoulder joints and other joints in the body are being replaced as well.

Existing joint prostheses generally have a protruding, or male, member (sometimes called a “ball”) and a recessed, or female, member (sometimes called a “socket”). Initially, both the male and female members were made of metal. However, experience indicated that this metal-on-metal construction resulted in a high degree of wear in the earlier designs at locations where there was moving contact between the male member and the female member. (The locations where mating joint surfaces contact are herein called “bearing surfaces.”) This wear on bearing surfaces caused early artificial joint failures, and usually mandated additional surgery to go back into the body and replace the worn artificial joint with a fresh, new artificial joint.

In order to reduce wear on the joint components, the female component was modified to include a non-metallic (usually plastic) insert, which covered the bearing surface of the female component. This has resulted in artificial joints that have metal-on-plastic contact, which has been the prevalent artificial joint design for some time now. One example of an artificial joint that utilizes dissimilar materials at the bearing surfaces is presented in U.S. Pat. No. 5,879,407 to Waggener. In Waggener an artificial joint includes a ball and a socket. The surface of the ball is ceramic and the surface of the socket is made of a noble metal or alloy, such as platinum. An example of a more common plastic-on-metal artificial joint is shown in U.S. Pat. No. 4,055,862 to Farling.

In metal-on-plastic joints, considerable research has been directed toward improving the characteristics of the plastic insert so that its moving contact with the metal surface of the mating artificial joint component would undergo minimal wear in normal use. Notwithstanding some advances in the area of metal finishes, as well as in the area of polishes for the plastic insert, the metal-on-plastic design still has significant wear and tends to fail over time due primarily to wear. More particularly, there are two distinct concerns about wear. One concern is that the bearing surface will wear out completely, such as when a plastic insert bearing surface wears out and exposes an underlying stem. Another concern is that particles that wear off of the joint may remain in the vicinity of the joint and cause problems such as increased wear and loosening of the insert or prosthesis.

### SUMMARY OF THE INVENTION

The inventor of the present invention has recognized a problem inherent in the above described artificial joint research. Specifically, the research appears to be fixated on metal-on-plastic artificial joints. The possibility of plastic-on-plastic artificial joints appears to have been neglected or rejected as unfeasible.

According to the present invention, and ignoring the conventional wisdom that artificial joints should be metal-on-metal or plastic-on-metal, it has been determined that plastic-on-plastic artificial joints are feasible, and are sometimes superior to conventional plastic-on-metal designs. This is especially true for plastic-on-plastic joints where the plastic material(s) are chosen to have superior wear characteristics. Generally speaking, less wear means fewer artificial joint failures and associated artificial joint replacement surgeries.

More subtly, wear resistant artificial joints made according to the present invention may facilitate the manufacture of artificial joints that precisely emulate the complex bearing surface geometry of a healthy joint. This is good because it allows the body to move in a natural fashion and will limit range of movement less. Also, if more precise emulation of healthy joint geometry is achieved, the reduced wear of the present invention can help the joint maintain this geometry over time. For example, if a normal joint bearing surface has a narrow ridge, the plastic-on-plastic artificial joint of the present invention may be made to more easily form the narrow ridge in the first place, and to also allow the narrow ridge to hold out against wear over time.

According to a preferred aspect of the present invention, ultra high molecular weight polyethylene (“UHMWPE”) is used on both mating, bearing surfaces of an artificial joint. This UHMWPE-on-UHMWPE joint experiences very little wear and represents a tremendous advance over conventional metal-on-plastic joints. The decrease in artificial joint wear on artificial joints made according to the present invention may mean fewer replacement surgeries and diminished surgery-related health risks.

At least some embodiments of the present invention have one or more of the following advantages, objects and/or benefits:

(1) to provide an artificial joint that exhibits less wear in normal use;

(2) to provide an artificial joint that less frequently experiences catastrophic or semi-catastrophic failure in normal use;

(3) to provide an artificial joint that generates less debris at its bearing surface;

(4) to provide an artificial joint that requires less frequent (surgical) replacement;

(5) to provide an artificial joint that is easier to move;

(6) to provide a less expensive artificial joint;

(7) to provide an artificial joint that can be more easily manufactured and sculpted to better match the shape of the cartilaginous surface of a healthy joint; and

(8) to provide an artificial joint that can better maintain the (sometimes intricate) shape of the cartilaginous surface of a healthy joint, notwithstanding wear caused by normal use of the artificial joint.

According to the present invention, an artificial joint includes a first joint member and a second joint member. The first joint member includes a first bearing surface made of artificial, non-metallic material. The second joint member includes a second bearing surface made of artificial, non-metallic material. The first bearing surface and the second bearing surfaces are shaped to mate to guide relative movement of the artificial joint.

According to another aspect of the present invention, an artificial human knee joint includes a first joint member and a second joint member. The first joint member includes a first bearing surface made of artificial material. The second joint member includes a second bearing surface made of artificial material. The first bearing surface and the second bearing surfaces are shaped to mate to guide relative movement of the artificial joint. The first and second bearing surfaces have a coefficient of friction of less than 0.1 when the joint is placed under normal use in a human body.

According to another aspect of the present invention, an artificial joint includes a first joint member, which includes a first bearing surface and a first anchor member. The first anchor member has a generally tubular shape, and includes an inner anchor surface and an outer anchor surface. The inner anchor surface is disposed around an interior surface of the first anchor member. The outer anchor surface is disposed around an exterior surface of the first anchor member.

Other advantages, objects and benefits will become apparent through a review of the rest of this document.

### BRIEF DESCRIPTION OF THE DRAWINGS

The present invention will become more fully understood from the detailed description given below, together with the accompanying drawings, which are given by way of illustration only, and thus are not to be construed as limiting the scope of the present invention. In the drawings:

FIG. 1 is a perspective view of a first embodiment of an artificial joint according to the present invention;

FIG. 2 is a cross-sectional view of the first embodiment of an artificial joint according to the present invention;

FIG. 3 is a cross-sectional view of a second embodiment of an artificial joint according to the present invention;

FIG. 4 is a perspective view of a third embodiment of an artificial joint according to the present invention;

FIG. 5 is a cross-sectional view of the femoral component of the third embodiment artificial joint; and

FIG. 6 is another cross-sectional view of the femoral component of the third embodiment artificial joint.

### DESCRIPTION OF THE PREFERRED EMBODIMENTS

Before starting a description of the Figures, some terms will now be defined.

Definitions

the present invention: at least some embodiments of the present invention; references to various feature(s) of the “present invention” throughout this document do not mean that all claimed embodiments or methods include the referenced feature(s).

ultra high molecular weight polyethylene (“UHMWPE”): any polyethylene resin with a molecular weight greater than 1,000,000.

“at least double melt”: material that has undergone double melting, triple melting or any greater number of melts.

“at least triple melt”: material that has undergone triple melting or any greater number of melts.

bone: can include calcic matter, as well as cartilaginous matter, marrow and any other type of matter included in a bone structure.

coefficient of friction: refers to the specific coeffecient of friction value as normally measured in the context of artificial joints.

open-ended interior space of an artificial joint component: refers to interior space that is open ended at the time the artificial joint is inserted in a bone.

Artificial: means material not made by a body at the location where the material is present in the body; for example, plastic is an artificial material because it is not manufactured by a body at all; as a further example, if material is taken out of a person's left knee and inserted into their right knee, that material would be artificial, for purposes of this document, because the material, while manufactured by the body, is not located at the location where the body manufactured it.

To the extent that the definitions provided above are consistent with ordinary, plain and accustomed meanings (as generally evidenced, inter alia, by dictionaries and/or technical lexicons), the above definitions shall be considered supplemental in nature. To the extent that the definitions provided above are inconsistent with ordinary, plain and accustomed meanings (as generally evidenced, inter alia, by dictionaries and/or technical lexicons), the above definitions shall control. If the definitions provided above are broader than the ordinary, plain and accustomed meanings in some aspect, then the above definitions will control at least in relation to their broader aspects.

To the extent that a patentee may act as its own lexicographer under applicable law, it is hereby further directed that all words appearing in the claims section, except for the above defined words, shall take on their ordinary, plain and accustomed meanings (as generally evidenced, inter alia, by dictionaries and/or technical lexicons), and shall not be considered to be specially defined in this specification. Notwithstanding this limitation on the inference of “special definitions,” the specification may be used to evidence the appropriate ordinary, plain and accustomed meanings (as generally evidenced, inter alia, by dictionaries and/or technical lexicons), in the situation where a word or term used in the claims has more than one pre-established meaning and the specification is helpful in choosing between the alternatives.

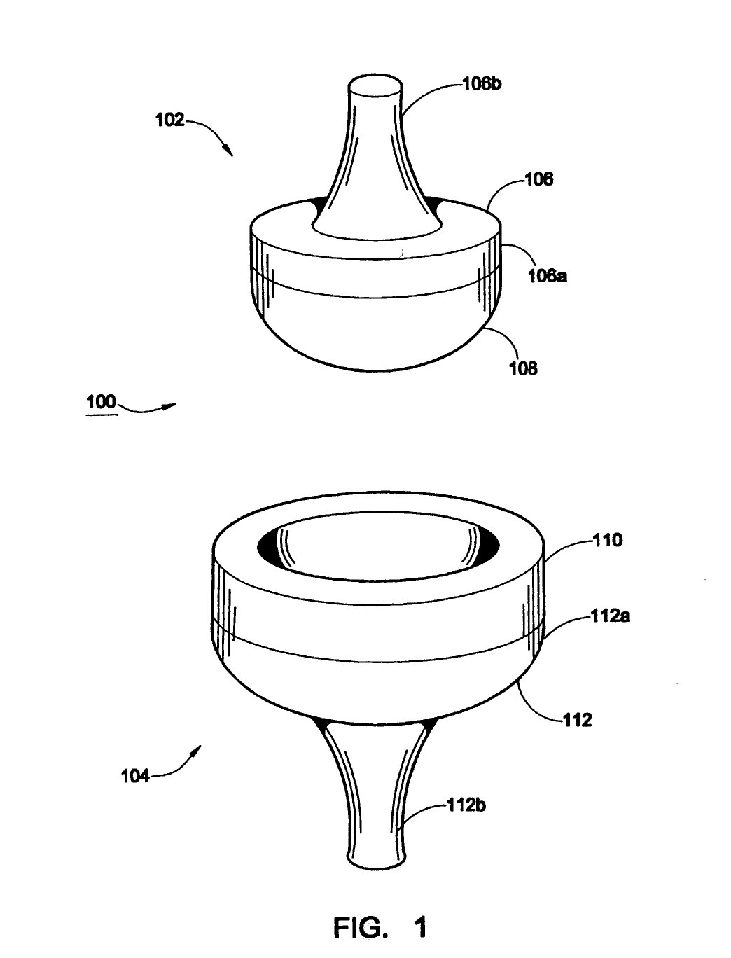

Referring to FIGS. 1 (perspective view) and 2 (cross-sectional view), an artificial joint 100 is shown. Artificial joint 100 is a ball and socket joint that allows rotation in more than one plane. Artificial joint 100 includes a ball component 102 and a socket component 104. Ball component 102 includes base plate 106 and ball insert 108. Base plate 106 includes base portion 106a and stem portion 106b. When artificial joint 100 is inserted in a body, at least a portion of stem 106b is driven into the subconjural portion of one of the bones of the joint in the conventional manner.

Base plate 106 is preferably made of metal and is preferably made of titanium or stainless steel. Alternatively, base plate 106 could be made of other metals and alloys to achieve the desired characteristics and functionality. For example, the same metals which are used to make the stem portion of conventional plastic-on-metal joints can be similarly used to make base plate 106. Ball insert 108 is preferably made of UHMWPE and is adhered to base plate 106 in the manner conventionally used to adhere polyethylene and metal parts in conventional plastic-on-metal artificial joints. The preferred construction of ball insert 108 will be more fully described below.

Socket component 104 comprises a base plate 112 and a socket insert 110. Base plate 112 includes base portion 112a and stem portion 112b. When artificial joint 100 is inserted in a body, at least a portion of stem 112b is driven into the subconjural portion of one of the bones of the joint in the conventional manner. Base plate 108, like base plate 106, is preferably made of titanium or stainless steel.

When both of stem 106b and stem 112b are properly driven into adjacent bones, ball insert 108 and socket insert 110 contact to become bearing surfaces, whose shapes guide permitted motion of the artificial joint. Socket insert 110 is preferably made of UHMWPE and is adhered to base plate 112 in any manner conventionally used to adhere polyethylene and metal parts in conventional plastic-on-metal artificial joints, such as by gluing, bonding, screwing, or press-fitting.

An important difference between at least some embodiments of the present invention and conventional artificial joints is that neither of the bearing surfaces 108, 110 are made of metal. Rather, the bearing surfaces are both non-metallic. This is because metal bearing surfaces wear (as in a metal-on-metal joint) or cause wear (as in a plastic-on-metal joint). This use of a metal bearing surface is generally inconsistent with the extremely low characteristics of friction, which are believed to be achievable with the present invention.

More preferably, the bearing surfaces are both plastic, and even more preferably, both bearing surfaces are polyethylene. Polyethylene's combination of strength, toughness, smoothness, compressibility, elasticity, stiffness, pore density and other material properties is well-known, and polyethylene is conventionally preferred in plastic-on-metal artificial joints. However, when polyethylene constitutes both bearing surfaces, as in the present invention, the relevant coefficient of friction and associated wear is believed to decrease dramatically. It is believed that certain other plastics could also lead to similar advantageous results.

The preferred polyethylene bearing insert surfaces of the present invention is UHMWPE. It is believed that the use of UHMWPE can lead to a relevant coefficient of friction of 0.1, 0.05 or even as low as 0.01. With coefficients of friction this low, there is drastically reduced wear of the bearing surface. Reduced wear of the bearing surface means that the bearing surface will maintain its shape and integrity, and that there will be less debris and associated particle wear. It is believed that the low friction artificial joints of the present invention may substantially outlast a human or animal lifetime so that surgeries to replace worn or particle-damaged artificial joints will no longer be necessary.

Another preferred type of polyethylene according to the present invention is polyethylene that has been melted down two or more times. Multiple melt polyethylene generally exhibits increased purity, cross-linking, elasticity, and strength, and a decreased tendency to delaminate. Accordingly, multiple melt polyethylene has been shown to be capable of high repetition translational contact with an equivalent polyethylene product to produce a virtually wearless system. Double melt polyethylene is therefore preferred according to the present invention. Triple (or more) melt polyethylene is even more preferred. One brand of this multiple-melt polyethylene is known as Dursul. (It is noted that the word Dursul may be subject to trademark rights.)

All artificial joint components are preferably sterilized in any conventional manner. The inserts are preferably sterilized by gamma radiation.

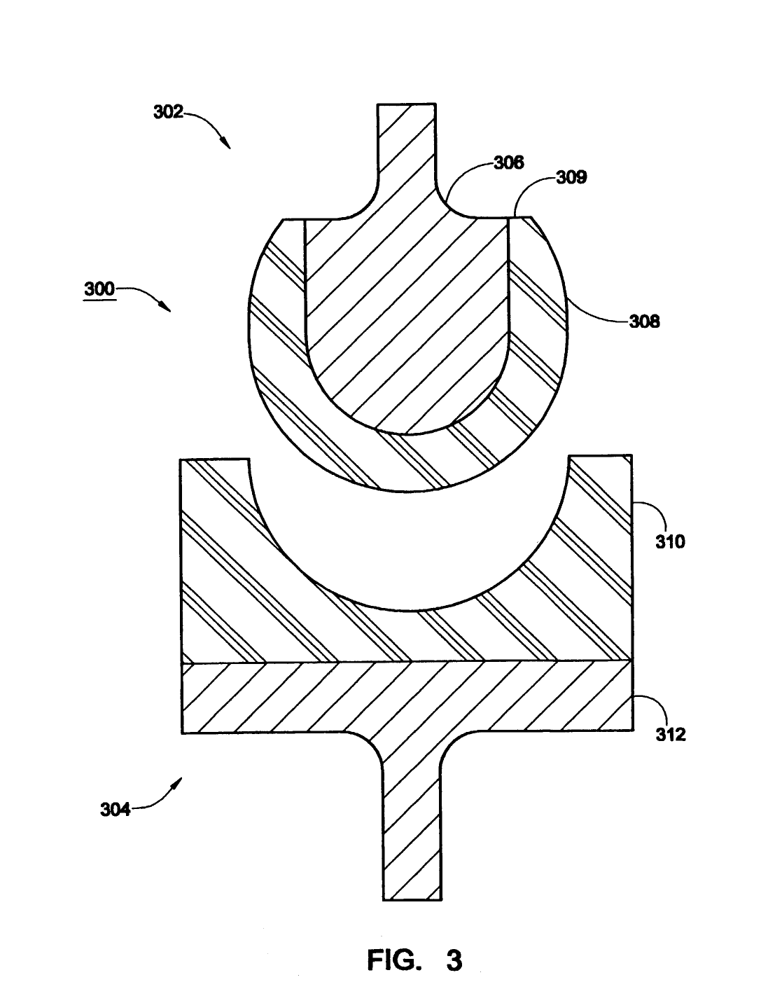

Referring now to FIG. 3, an alternative embodiment of an artificial joint 300 is shown. Artificial joint 300 includes ball component 302 and socket component 304. Ball component 302 includes base plate 306 and ball insert 308. Socket component 304 includes base plate 312 and socket insert 310. Both ball insert 308 and socket insert 310 are made of triple melt UHMWPE. As shown in FIG. 3, ball insert 308 completely surrounds base plate 306, in the vicinity of reference numeral 309, for improved wear characteristics at both ball component 302 and socket component 304.

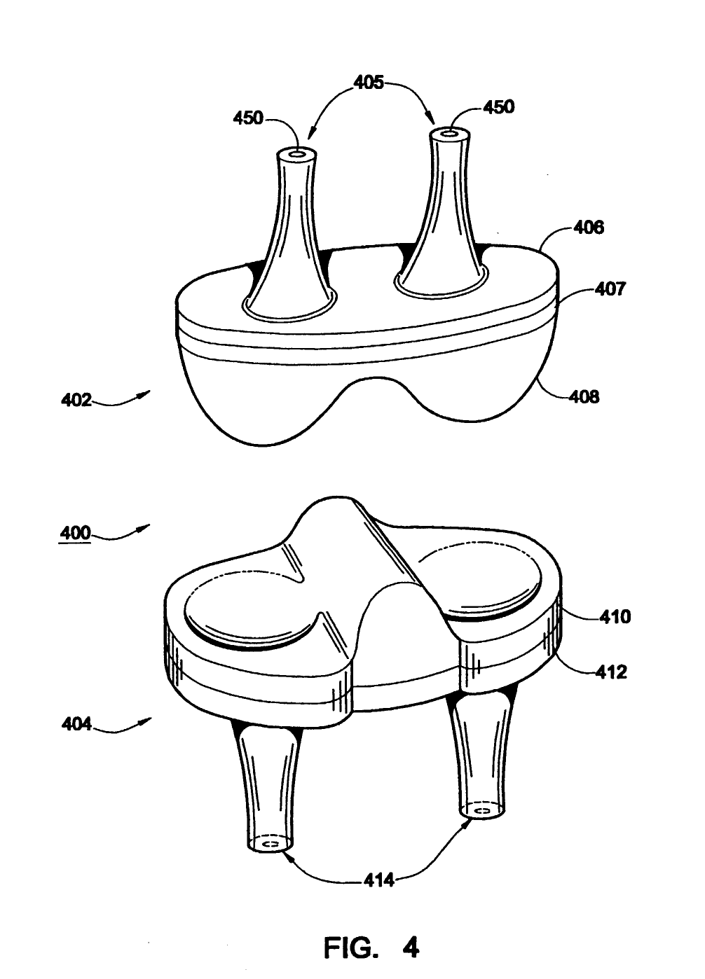

Referring now to FIG. 4, artificial knee joint 400 is shown. Knee joint 400 includes femoral component 402 and a tibial component 404. Femoral component 402 includes metal base plate 406 and triple melt UHMWPE insert 408. Similarly, tibial component 404 includes metal base plate 412 and triple melt UHMWPE insert 410. Metal base plate 406 includes anchor (or stem) members 405 for insertion into the subconjural bone of the femur. Metal base plate 412 includes anchor members 414 for insertion into the subconjural bone of the tibia. The mating, bearing surfaces of inserts 408 and 410 are preferably shaped to match the natural geometry of a healthy knee as closely as possible.

In order to form this geometry, the inserts may be formed by any conventional UHMWPE forming technique, such as by molding and/or machining. When choosing the forming technique, the ability of the forming technique to accurately achieve the desired shape should be considered. The effect that the forming technique may have on surface wear should also be considered. For example, it is believed by some that molded polyethylene bearing surfaces have better wear characteristics than machined bearing surfaces, at least in the context of conventional plastic-on-metal joints.

It is further noted that the complex geometry shown in FIG. 4, which attempts to accurately emulate the shape of cartilage in a healthy knee, includes ridges and depressions. It is believed that the plastic-on-plastic joints of the present invention may be especially advantageous in the context of knee joint replacements because the complex geometry of the artificial knee joint will hold its shape better under decreased friction and wear. For example, the anatomy of a natural knee can be duplicated all the way to the periphery of the cortical rim, such that the artificial joint feels and performs more naturally. Also, artificial knee joints are subjected to a high degree of stress and associated wear because they carry most of the body's weight and are subjected to both intermittent and repetitive motions. Therefore, it is especially advantageous to decrease wear in joints like the knees and hips, which tend to wear faster than lower stress joints.

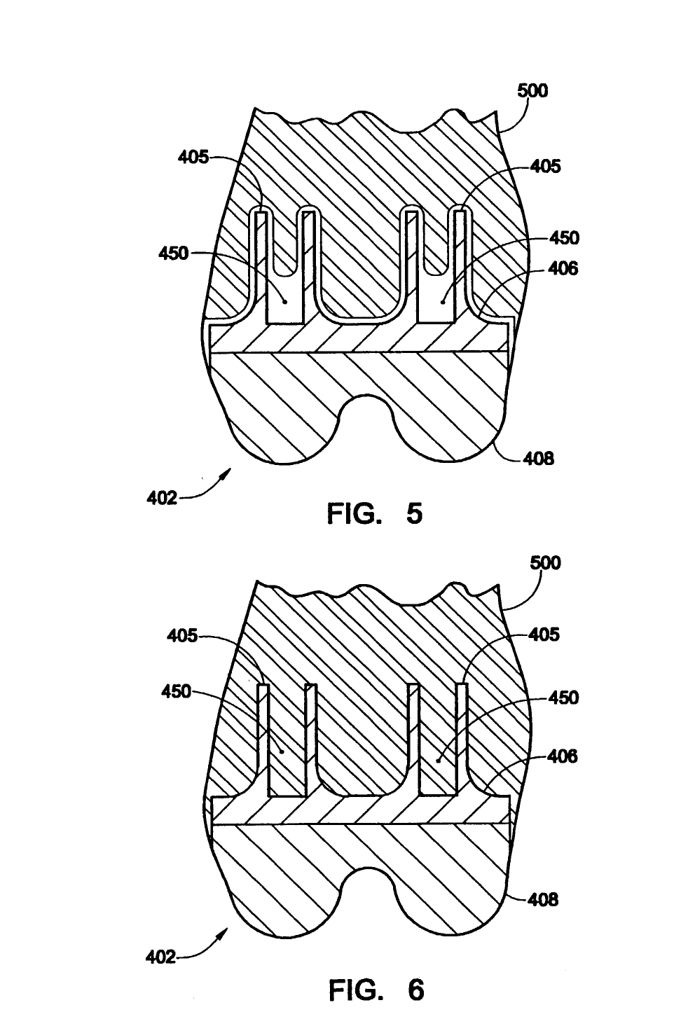

Anchor members 405 will now be discussed in more detail with reference to FIGS. 5 and 6. FIG. 5 shows femoral component 402 just after it has been driven into a femur bone 500. As is conventional, material may be removed from the end of the femur bone prior to insertion of femoral component 402. It is the anchor members 405 that secure femoral component 402 to femur 500.

While conventional anchor members are cylindrically solid in construction, anchor members 405 are tubular in construction so that they include an open-ended interior space 450. As shown in FIG. 5., bone matter begins to flow into this interior space when it is first driven into femur 500.

As shown in FIG. 6, as time passes, the bone grows to fill this interior space 450, and bonds to the interior wall of interior space 450. By filling interior space 450 and bonding to it, the bond between femur 500 and femoral component 402 is greatly strengthened as the bone heals. This is advantageous because the more secure attachment allows the artificial joint to perform better, and can ward off repair work that is conventionally necessitated when a prosthetic component becomes loose. Preferably, the inside of the interior space is constructed and treated to promote bonding with the regrowing bone.

While anchor members 405 are cylindrically tubular in shape, other tube profiles are possible. Also, non-tubular constructions with other types of interior spaces are also possible, such as open ended or hollowed out spheroid anchors (although these may be more difficult to drive into the bone). Furthermore, ridges or other surface discontinuities can be provided on the interior anchor member surface to further promote tight bonding between bone and prosthesis.

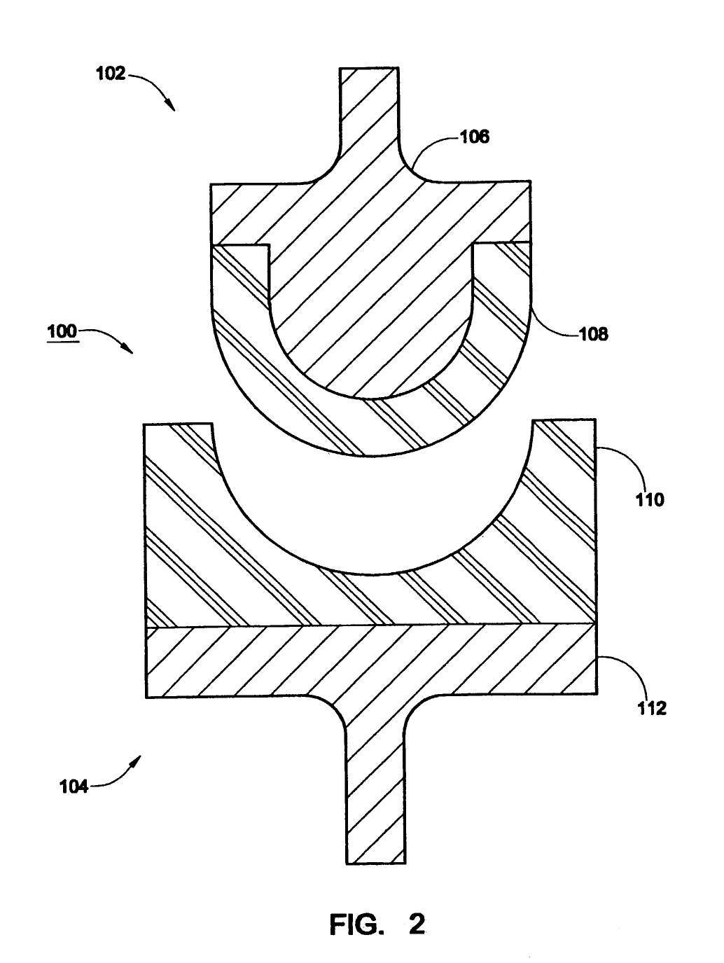

Although the artificial joints of the present invention have been explained in terms of a generic ball and socket joint geometry of FIGS. 1 to 3 and a knee joint of FIG. 4, those skilled in the art will appreciate that the range of joints for which the present invention can be utilized is significant. For example, the present invention could be utilized in not only a replacement knee, but could equally be utilized to replace the joints in ankles, feet, hips, elbows, wrists, hands, shoulders or mouth, as well as other joints in a human or animal. Also, while joints according to the present invention are preferably applied to joints that emulate natural joint geometry, the non-metallic bearing surfaces and tubular stems of the present invention could also be applied in the context of joints that do not precisely emulate a natural joint. For example, U.S. Pat. No. 3,945,053 (incorporated by reference) discloses an artificial, rolling contact knee joint which has bearing surfaces that are not particularly similar to the natural bearing surfaces of a knee.

Another preferred embodiment of the present invention includes use of all poly male and female components with roughened and similar anchoring parts that are bonded to bone by bone cement (e.g., polymethyl-methacrylate based bone cement).

Many other variations on the above-described artificial joints are possible. Such variations are not to be regarded as a departure from the spirit and scope of the invention, but rather as modifications intended to be encompassed within the scope of the following claims, to the fullest extent allowed by applicable law.

What is claimed is:

1. An artificial joint comprising:
a first joint member comprising a first bearing surface made of artificial, non-metallic material;
a second joint member comprising a second bearing surface made of artificial, non-metallic material, with the first bearing surface and the second bearing surfaces being shaped to mate to guide relative movement of the artificial joint.

2. The artificial joint of claim 1 wherein:
the first bearing surface is made of plastic; and
the second bearing surface is made of plastic.

3. The artificial joint of claim 2 wherein:
the first bearing surface is made of polyethylene; and
the second bearing surface is made of polyethylene.

4. The artificial joint of claim 3 wherein:
the first bearing surface is made of ultra high molecular weight polyethylene; and
the second bearing surface is made of ultra high molecular weight polyethylene.

5. The artificial joint of claim 3 wherein:
the polyethylene of the first bearing surface is at least double melt polyethylene; and
the polyethylene of the second bearing surface is at least double melt polyethylene.

6. The artificial joint of claim 5 wherein:
the polyethylene of the first bearing surface is at least triple melt polyethylene; and
the polyethylene of the second bearing surface is at least triple melt polyethylene.

7. The artificial joint according to claim 2 further comprising:
a first bone, with the first joint member being constrained to the first bone; and
a second bone, with the second joint member being constrained to the second bone.

8. The artificial joint of claim 2 wherein:
the first joint member further comprises a first anchor portion made of artificial material, the first anchor portion being structured to constrain the first joint member to a bone; and
the second joint member further comprises a second anchor portion made of artificial material, the second anchor portion being structured to constrain to second joint member to a bone.

9. The artificial joint of claim 8 wherein:
the first and second joint members are all poly; and
the first and second anchor portions are roughened.

10. The artificial joint of claim 4 wherein:
the first anchor portion is made of metal; and
the second anchor portion is made of metal.

11. An artificial human knee joint comprising:
a first joint member comprising a first bearing surface made of artificial material;
a second joint member comprising a second bearing surface made of artificial material, with the first bearing surface and the second bearing surfaces being shaped to mate to guide relative movement of the artificial joint, and with the first and second bearing surfaces having a coefficient of friction of less than 0.1 when the joint is placed under normal use in a human body.

12. The joint of claim 11 wherein the coefficient of friction is less than 0.01.

13. The joint of claim 11 wherein the first and second bearing surfaces are both made of triple melt ultra high molecular weight polyethylene.

14. An artificial joint comprising a first joint member comprising:
a first bearing surface; and
a first anchor member defining an open-ended interior space, the first anchor member comprising:
an inner anchor surface disposed around an interior surface of the interior space; and
an outer anchor surface disposed around an exterior surface of the first anchor member.

15. The artificial joint of claim 14 further comprising a bone, with the first anchor member being disposed within the bone so that at least a portion of the inner anchor surface is in contact with the bone.

16. The artificial joint of claim 15 wherein the first anchor member is disposed so that at least a portion of the outer anchor surface is in contact with the bone.

17. The artificial joint of claim 14 wherein the first anchor portion is made of metal.

18. The artificial joint of claim 17 wherein the first anchor portion is made of titanium.

19. The artificial joint of claim 14 wherein the inner anchor surface comprises surface discontinuities structured to promote bonding of the inner anchor surface to growing bone.

20. The artificial joint of claim 14 further comprising a second joint member comprising:
a second bearing surface shaped to mate with the first bearing surface, with the first and second bearing surfaces both being made of ultra high molecular weight polyethylene; and
a second anchor member defining an open-ended interior space, the second anchor member comprising:
an inner anchor surface disposed around an interior surface of the interior space; and
an outer anchor surface disposed around an exterior surface of the first anchor member.
